(19) United States
(12) Patent Application Publication
(10) Pub. No.: US 2009/0261179 A1
(43) Pub. Date: Oct. 22, 2009

(54) BOTTLE CAP SEAL FOR WICKED AIR FRESHENER

(75) Inventor: Lynn A. Hall, Lexington, KY (US)

Correspondence Address: WOOD, HERRON & EVANS, LLP, 2700 CAREW TOWER, 441 VINE STREET, CINCINNATI, OH, 45202, US

(73) Assignee: ASHLAND LICENSING AND INTELLECTUAL PROPERTY LLC, Dublin, OH (US)

(21) Appl. No.: 12/423,908

(22) Filed: Apr. 15, 2009

Related U.S. Application Data
(60) Provisional application No. 61/124,300, filed on Apr. 16, 2008.

(51) Int. Cl. A61L 9/12

(52) U.S. Cl. 239/44; 239/326; 239/602

(57) ABSTRACT

An air freshener replacement bottle having an open ended, externally threaded neck a wick holding sleeve which press fits onto the open end of the bottle. The sleeve has an outwardly directed flange with a lower face that abuts against an upper end of the bottle neck. An internally threaded cap is threaded onto the neck of the bottle compressing the wick holder flange forming a seal. The cap has a cylindrical wall smaller in diameter than the lower portion of the cap, protruding from its top. The cylindrical wall contains at least one annular groove on it outside. A wick extends through the sleeve and the cylindrical wall with one end projecting into the cavity of the bottle and the opposite end exposed to the air. Another cap whose internal diameter is sized slightly larger than the cylindrical wall on the first cap has at least one internal annular rib sized slightly smaller than said at least one groove in said cylindrical wall. The second cap is therefore snapped onto the first cap to seal the wick to prevent evaporation with the ribs and grooves providing a tight seal.

### RELATED APPLICATION

This application is related to and claims the benefit of U.S. Provisional Patent Application Ser. No. 61/124,300, filed Apr. 16, 2008, the disclosure of which is incorporated herein by reference.

### TECHNICAL FIELD

The present invention relates to air freshener or fragrance replacement bottles and in particular to providing a means of sealing the bottle for storage, before use in an air freshener housing thus preventing waste of the liquid due to leakage and evaporation.

### BACKGROUND OF THE INVENTION

Air fresheners are commonly used in homes, offices, automobiles, etc. A typical air freshener unit is composed of a housing which clips onto the outlet of an air conditioner or ventilation grill. The housing is designed to have a bottle of a desired fragrance inserted and held in place by, for example, a snap collar. The bottle has a wick exposed from its opening through the neck and disposed inside the housing in fluid communication with the liquid air freshener fragrance. Fragrance is delivered into the room or cabin as air flows over the wick and fragrance vapors are pulled out of the bottle as liquid is evaporated in the air. Air must be able to displace the fluid flowing through the wick. Typically the wick is spaced apart from the neck of the bottle by a projection, groove, or other spacing means in order for air to displace the fluid during use.

It has been seen in the market that there is a problem with shelf life and shipment of the replacement air freshener due to leakage or evaporation of the liquid around the cap and wick holder area of the replacement bottle. Due to the expansion and contraction of the fluid during shipment over long periods of time and hot and cold environments, liquid from the bottle will tend to seep up around the wick area. Wicked air freshener caps are typically composed of a wick holding member having an insertable cylindrical portion disposed into the neck of the bottle and having a peripheral flange or collar extending over the edge of the bottle neck. A threaded cap retaining member having an opening in the top usually cooperatively engages a cap having a peripheral flange. Screwing the cap retaining member cooperatively engages threads projecting from the top distal end portion of the bottle neck and holds the cap flange against the top surface of the wick retaining member thereby providing a seal.

Exposure to heat and cold may form a vacuum area beneath the cap drawing liquid from the wick under certain conditions resulting in a small quantity of liquid having a concentrated fragrance leaking out of the bottle from around the cap and wick area upon first opening the bottle. The person opening the bottle may spill the contents or at least get the liquid on their hands.

There are numerous examples of fragrance delivery systems using replaceable containers with simple wicking systems. U.S. Pat. No. 6,446,880 B1 by Schram et al., issued Sep. 10, 2002, for REPLACEABLE RESERVOIR FOR AN ATOMIZING DEVICE discloses a replaceable bottle whose sealing system is a rubber type overcap which slides over the bottle opening tightly. U.S. Pat. No. 5,909,845 by Greatbatch et al., issued Jun. 8, 1999, for WICK-BASED LIQUID EMANATION SYSTEM WITH CHILD RESISTANT OVERCAP discloses a similar tight fitting rubberized cap which snaps onto the bottle opening. These sealing systems depend on the elastic quality of the rubberized cap and its fit onto the mouth of the bottle. They do not embody surfaces which are shaped to mate with one another and compressibly engage when a threaded cap is screwed tightly forming a better seal.

Other prior art includes: U.S. Pat. No. 6,293,474 B1 by Helf et al., issued Sep. 25, 2001, for DELIVERY SYSTEM FOR DISPENSING VIOLATILES; U.S. Pat. No. 5,038,394 by Hasegawa et al., issued Aug. 6, 1991, for THERMAL VAPORIZOR; and U.S. Pat. No. 6,010,333 by Tendick, issued Jan. 4, 2000, for REFILLABLE BURNER ASSEMBLY.

### SUMMARY OF THE INVENTION

Therefore, it is an object of the invention to provide a replacement fragrance bottle containing a partially exposed wick for fragrance delivery, intended to be held within a housing clipped onto a ventilation grill, which has an improved shelf life due to a better sealing system integrated into the bottle cap.

It is also an object of the invention to provide a replacement fragrance bottle containing a partially exposed wick for fragrance delivery wherein a removable cap may be snapped onto the bottle in times when the user decides to discontinue its use and save the fragrance for later use.

A new design for an air freshener replacement bottle system which will have a longer shelf life is presented herein. The system includes a bottle with an externally threaded neck, a wick retained by a wick holder containing a flange which acts as a stop when pushing the wick holder into the bottle and also as a sealing surface in contact with the cap and the top of the bottle, a first cap having internal threads which is threaded onto the neck of the bottle, and a second cap snaps onto a cylindrical wall portion integral with the first cap extending upward from the top of the first cap section and forming tight seal.

The first cap is threaded onto the bottle after inserting the wick and the wick holder. The wick holder flange is sealingly captured between the top lip of the bottle opening and an internal annular surface of the first cap section. Additionally, this first cap section has a cylindrical wall extending from its top through which the wick extends and compressibly captures and supports the wick. The cylindrical wall has an outer diameter which is less than the outer diameter of the first cap section. The exterior surface of the cylindrical wall has at least one annular rib extending outward forming a sealing surface or surfaces which communicate with at least one mating valley like surface on the inside of the second cap. These ribs compressibly snap into these valley surfaces to create an air tight fit as the second cap section is forcibly pushed onto the first cap section.

The objects and advantages of the present invention will be further appreciated in light of the following detailed description and drawings in which:

### BRIEF DESCRIPTION OF THE DRAWING

FIG. 1 is a perspective view of the bottle, wick and cap assembled;

FIG. 2 is an exploded view of the present invention; and

FIG. 3 is a cross-sectional view of the upper portion of the present invention.

### DETAILED DESCRIPTION

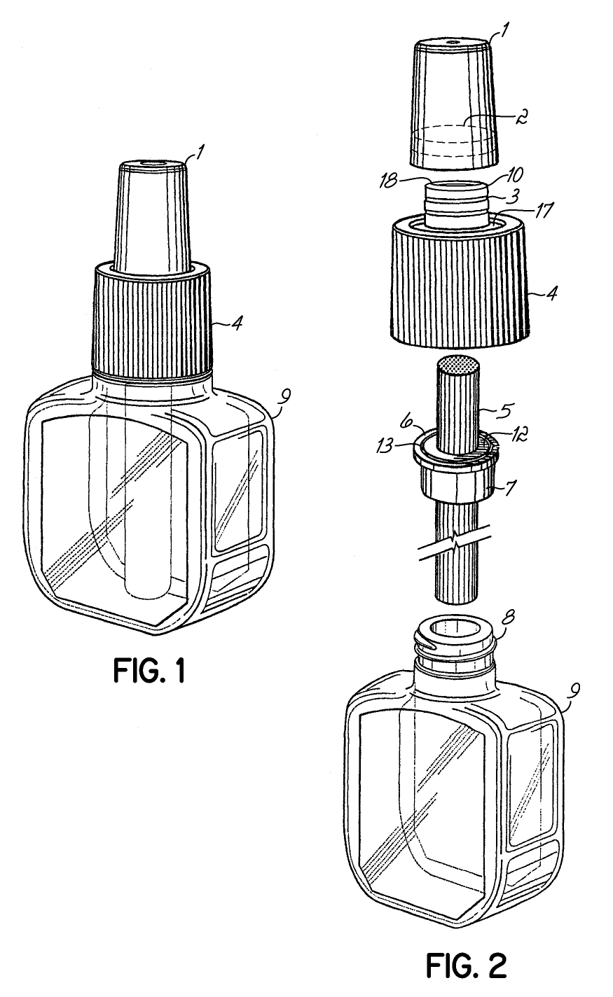

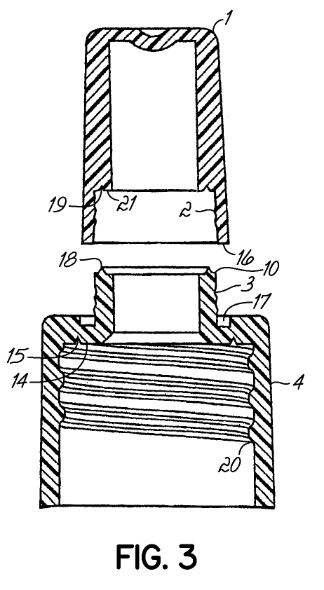

Illustrated in FIG. 2 is a bottle 9 having an open ended, externally threaded neck 8. A wick holding sleeve 7 press fits into the neck of the bottle, through the open end thereof. Sleeve 7 includes an outwardly directed flange 6 with a lower face that abuts against an upper end of the bottle neck 8. A wick 5 extends through a central opening in sleeve 7 and has one end thereof projecting into the cavity of the bottle 9 and an opposite exposed end.

An internally threaded first cap 4 threads onto the neck 8 of bottle 9 compressing the wick holder flange 6 adjacent the neck and forming the seal. Sleeve 7 includes a planar surface 12 with a ridge 13 which forms an additional seal with a corresponding groove 14 on the bottom surface 15 of first cap 4. A cylindrical wall or neck 10 extends from the top of and is an integral part of the first cap 4. The upper portion of the wick 5 extends upward out of and is supported by the cylindrical wall 10. The lower section of the wick 5 extends down to the bottom of the bottle to wick the fragrant liquid up into upper wick portion and into the air.

A second cap 1 includes one or more inwardly extended annular ribs 2. Annular grooves 3 are molded in the outside of the cylindrical wall 10 of cap 4. The second cap 1 is adapted to be pressed onto the cylindrical wall 10 of first cap 4 with the ribs 2 snapped into the grooves 3, forming an airtight seal, preventing unwanted evaporation and waste of the fragrant liquid. When cap 1 is snapped onto cylindrical wall 10, the bottom edge 16 of second cap 1 rests in an annular groove or channel 17 at the base of cylindrical wall 10. This channel assists in providing a seal and acts as a reservoir if any fluid leaks from wick 5.

Further, cylindrical wall 10 includes a ridge 18 which seats in a groove 19 on the inner surface 21 of cap 1. This forms an additional seal. The second cap 1 can be removed for use and then replaced to save the product.

Caps 1 and 4 with the two mating surfaces and the ribs 2 and grooves 3 can be composed of any combination of rubber, various elastomer compositions, plastics, or any composition found to provide a tight and long lasting seal which does not in any way react with the stored liquid fragrance.

In use, the air freshener bottle 9 is filled or partially filled with air freshening liquid and the sleeve 7 with wick 5 positioned within the sleeve is forced into the neck 8 of bottle 9. Primary cap 4 is then threaded onto the neck 8. The bottom surface 14 of cap 4 presses against the planar surface 12 of wick holder 7 with flange 6 pressed against neck 8 of bottle 9. In turn, the ridge 13 on planar surface 12 engages the groove 15 on the inner surface 14 of cap 4, forming a tight seal.

The secondary cap 1 is then press fitted onto the cylindrical wall 10 of cap 4 with the annular ribs 2 on the inner surface of cap 1 engaging the annular grooves 3 on the inside of cylindrical wall 4. This forms a further seal. Further, the bottom edge 16 of the cap 1 rests in annular groove 17 on the base of neck 10 further forming a seal. Thus, the present invention provides several redundant seals to ensure that no liquid escapes during storage.

For use, the cap 1 is simply pulled off the neck 10 of cap 4 exposing the wick 5, which in turn exposes liquid within the wick to the atmosphere causing it to evaporate and provide its intended effect. If any liquid should leak, it will collect in groove 17 until it evaporates.

The foregoing detailed description is given primarily for clearness of understanding and no unnecessary limitations are to be understood therefrom, for modifications will become obvious to those skilled in the art based upon more recent disclosures and may be made without departing from the spirit of the invention and scope of the appended claims.

This has been a description of the present invention along with the preferred method of practicing the present invention. However, the invention itself should only be defined by the appended claims, WHEREIN I CLAIM:

What is claimed is:

1. A bottle of air freshener comprising:
an open ended, externally threaded neck;
a wick holding sleeve press fitted into the neck of said bottle;
said sleeve having an outwardly directed flange with a lower face that abuts against an upper end of said neck;
a first internally threaded cap threaded onto said neck compressing said flange against said neck forming a seal;
said first cap having a cylindrical wall containing at least one annular groove on an outer surface of said cylindrical wall;
a wick extending through said sleeve and said cylindrical wall with one end thereof projecting into the cavity of the bottle and an opposite exposed end; and
a second cap having an internal diameter slightly larger than said cylindrical wall, said second cap containing at least one internal annular rib adapted to engage said at least one annular groove of said cylindrical wall.

2. The bottle claimed in claim 1 wherein said first cap includes an annular channel at a base of said cylindrical wall.

3. The bottle claimed in claim 2 wherein said second cap has a bottom edge fitted into said annular channel.

4. The bottle claimed in claim 1 wherein said sleeve includes a planar surface inward of said flange said surface compressed against a bottom inner surface of said first cap.

5. The bottle claimed in claim 4 wherein said cylindrical wall has an upper ridge adapted to engage a lower inner surface of said second cap.
